SEQUENCE LISTING (11 sequences)

SEQ ID NO: 1 - DNA, 800 bp, Homo sapiens
gtttagcgttgtttgttttttttatttttttttttcgaagttttttttggttcgaggaggtggcgagtgatgttttaggggtttttgagtgttttttttcgggttcgttagttttatacgtttatttcgcgggcgttttattgggcgtatcgtattgtgaatgtagtttcgggggtttttcgcggtttcgttttcgggggggttttatagcgtttttaagtggcggtcgtttaggtttcgcgggttttatttttcgttcgtatttcgttccggttagtttttttcgtttttttgttttcgttttatttgtttcgttgattgttggtttcgttagttttgttagttttacgtttttgtcgtttttattttcgttcgcgttttatttttttgcgcgattcgcgttcgttggtttttttttttttttttcgcgttttgtttgttttttttttttgttttcgtaggttttttggtatttaggtcgggaggcgacgcgtttagtcgtttaaacgggaatagttttggttgagggagttgtagcgtagtagagtatttgacggcgttaggttgcgtaggtgcggtacgaggagttttttcggtagcgaggaggttttgagtagtatggttcggaggagcgttttttttgtcgtcgcgttttggttttggagtatttttttgtgtttgttggtattgcgggcggaggtcgggtcgtcgtaggaggagagtttgtatttatggatcgatgtttattaggtaagagtatttataggtaaggttttcgtttttagcgtttttttttgttt

SEQ ID NO: 2 - DNA, 267 bp, Homo sapiens
cgtttaaacgggaatagttttggttgagggagttgtagcgtagtagagtatttgacggcgttaggttgcgtaggtgcggtacgaggagttttttcggtagcgaggaggttttgagtagtatggttcggaggagcgttttttttgtcgtcgcgttttggttttggagtatttttttgtgtttgttggtattgcgggcggaggtcgggtcgtcgtaggaggagagtttgtatttatggatcgatgtttattaggtaagagtatttatag

SEQ ID NO: 3 - DNA, 69 bp, Homo sapiens
tttgacggcgttaggttgcgtaggtgcggtacgaggagttttttcggtagcgaggaggttttgagtagt

SEQ ID NO: 4 - DNA, 20 bp, Homo sapiens
tatacgtttatttcgcgggc

SEQ ID NO: 5 - DNA, 21 bp, Homo sapiens
acgacgacccgacctccgccc

SEQ ID NO: 6 - DNA, 27 bp, Homo sapiens
cgtatttcgttcgcgttagtttttttc

SEQ ID NO: 7 - DNA, 21 bp, Homo sapiens
ctctcctcctacgacgacccg

SEQ ID NO: 8 - DNA, 19 bp, Homo sapiens
tttgacggcgttaggttgc

SEQ ID NO: 9 - DNA, 24 bp, Homo sapiens
actactcaaaacctcctcgctacc

SEQ ID NO: 10 - DNA, 16 bp, Homo sapiens
cggtacgaggagtttt

SEQ ID NO: 11 - DNA, 1290 bp, Homo sapiens
aaagtgcagggattacaggcgtgagccatcgcgcccggccgaattcagcaacttttaaaaaatatcagcaaacgtgaagatatccacgatgttagaggagccctaccccggagggtcaggtacagcttcgtccagggcccagggcactcttccgggcactgccgggttatcagggagacagacgggaatccccaaatgctgggtgtcgggcaagtaccagctggacgccctgcgcctcgagccaaggccagcgctgccatcggcaccatcggacagtcgagcctcgagttttaactgcttgggagcgcgcaaagtgccagcctatcgcagaacggagcgcatagggttggcggagagaggaatcctactggctgaaagggagacgaaggcaatttgcgccttcagtgagcgccggaggaggaacaggagtcatcacctcatcatcatcatcatcatcatcatcaccatcaccatcaccatcatcagcactcagtcaagcccagcgttgtctgcttccccatttccctcccccgaagcctcccttggcccgaggaggtggcgagtgatgtcccaggggtctctgagtgcccttctccgggtccgccagccctacacgcccacttcgcgggcgctccactggggcaccgcactgtgaatgcagcctcgggggtccctcgcggccccgcccccgggggggccccacagcgcccccaagtggcggccgcccaggcctcgcgggccccactcctcgctcgcacctcgctcgcgcagcccttcccgctcttctgttctcgctctatttgccccgctgactgctggcctcgccagctttgccagtcttacgtctctgccgcccccactcccgcccgcgccccatcttcttgcgcgactcgcgccgctggtccccccctcctcctcccgcgtcctgcctgccccctcctcctgctctcgcaggctccttggcacccaggccgggaggcgacgcgcccagccgtctaaacgggaacagccctggctgaggggctgcagcgcagcagagtatctgacggcgccaggttgcgtaggtgcggcacgaggagttttcccggcagcgaggaggtcctgagcagcatggcccggaggagcgccttccctgccgccgcgctctggtctggagcatcctcctgtgcctgctggcactgcgggcggaggccgggccgccgcaggaggagagcctgtacctatggatcgatgctcaccaggcaagagtactcataggtaaggcccccgcctccaggccttcccctgcct

(19) United States
(12) Patent Application Publication
(10) Pub. No.: US 2011/0129840 A1
(43) Pub. Date: Jun. 2, 2011

(54) METHOD, PROCESS, AND KIT FOR DIAGNOSIS OR PROGNOSIS OF COLORECTAL CANCER

(75) Inventors: Iradj Sobhani, Saint Maur (FR); Hicham Mansour, Oujda (MA)

(73) Assignee: Assistance Publique Hopitaux De Paris, Paris (FR)

(21) Appl. No.: 12/865,408

(22) PCT Filed: Jan. 23, 2009
(86) PCT No.: PCT/FR09/50101

§ 371 Date: Feb. 18, 2011

(30) Foreign Application Priority Data
Jan. 31, 2008 (FR) 0800543

(51) Int. Cl. C12Q 1/68; C07H 21/04

(52) U.S. Cl. 435/6.12; 435/6.14; 536/24.33; 536/24.31

(57) ABSTRACT

A process or method and a kit for the diagnosis/prognosis and follow-up of colorectal cancer, which includes a) the extraction of nucleic material from a biological sample, b) use of at least one pair of amplification primers in order to obtain amplicons of at least one target sequence, and c) use of at least one detection probe to detect the presence of the amplicons, the process or method including a modification step between steps 1) and b). Steps b) and c) are carried out at the same time. The target sequence is included in the WIF1 gene and the pair of primers includes at least one amplification primer including at least 15 nucleotide patterns of a nucleotide sequence selected from SEQ ID nos. 1 to 10 and/or the detection probe includes at least 15 nucleotide patterns of a nucleotide sequence selected from SEQ ID nos. 1 to 10.

This invention relates to a diagnosis/prognosis method, process and kit for detecting colorectal cancer for the purposes of diagnosis, prognosis or therapeutic follow-up.

More particularly, this invention is related to the amplification primers and hybridisation probes that may be used as part of the method and a diagnosis/prognosis kit for colorectal cancer.

“Diagnosis” means the determination of the affection of a person suffering from a given disorder; “prognosis” means the degree of seriousness and the subsequent development of a disorder and “therapeutic” relates to the curative treatment offered to the patient.

Colorectal cancer is the third most frequent cancer in humans and the second most frequent cause of death in France, and its prevalence is increasing incessantly every year.

Its cost for society depends on the stage of diagnosis and on the prognosis of the disease. In the very early stages or immediate precancerous stages, the disease is curable at a reduced cost, with no mortality. In the most advanced stages, its economic cost and mortality rate are very high.

The risk factors are partly due to our lifestyle and food habits, but also our genetic heritage. In general, hereditary colorectal cancers are characterised either by an abnormality of the APC gene or the Lynch syndrome, which leads to abnormalities of the DNA repair protein coding genes. In both cases, there have been either one or more gene mutations or one or more methylation abnormalities of these genes or the genes that regulate them.

From the histological standpoint, the mucous membrane that lines the colon and rectum develops aberrant crypt foci and adenomatous polyps that turn into invasive tumours. Hidden bleeding, which can be detected in the stools, may be a sign of the tumour when the adenomatous polyp becomes visible and at the most advanced stages. Such bleeding is intermittent.

However, on the genetic level, it is known that the element that triggers colorectal carcinogenesis is, from the aberrant crypt foci stage, the inactivation of the APC gene, the protein of which plays a role in proliferation control, apoptosis and the migration of intestinal epithelial cells. Such inactivation leads to the activation of certain signalling pathways, such as the WNT/beta catenin pathway, and the formation of early adenomas. The subsequent stages of colorectal carcinogenesis require the addition of other genetic abnormalities such as mutations of the K-RAS gene, the inactivation of the p53 gene or the inactivation of the genes involved in the DNA mismatch repair process (MMR gene).

The reduction of mortality due to this form of cancer calls for a diagnosis of the disease at the very early stages. The screening of asymptomatic persons aged above 50 is the best public health measure in the area. Currently, screening takes place through a biological test consisting in detecting hidden blood in the stools that may be biochemical (Gaiac, Magstream or Hemoccult) or immunological (Magstream, Eiken) followed by systematic colonoscopy if the test is positive, in order to detect and/or remove the tumours.

These tests are limited by their low sensitivities or specificities. The Hemoccult test is positive in 13 to 50% of cases with colon or rectum tumours, and at the same time, nearly 50% of the individuals with a positive Hemoccult result will go on to have normal colonoscopy results. The immunology test has improved the rate of sensitivity because of its quantitative approach to the search for faecal blood, but there is a risk of the specificity of the test diminishing if the positivity limit of the test is set very low, thus leading to unnecessary colonoscopies with a non-negligible impact on the cost to efficiency ratio.

That is why alternative tests are required. Epithelial colon cells include cells from tumours that are exfoliated every day in the stools. In spite of the low quantity of faecal human DNA, gene abnormalities due to tumours have the potential to become valuable markers in diagnostic testing.

A test based on the detection of the localised mutation of faecal DNA is available in the market. Its sensitivity is higher than those of other biological tests (search for hidden blood in the stools) but it is extremely expensive.

International patent application WO2005/112988 discloses compositions, processes and kits for diagnosing and treating cancers where the Wnt Inhibitory Factor1 (WIF1) is under-expressed. Compositions are provided to inhibit the proliferation of cancer cells, induce apoptosis in cancer cells by inhibiting Wnt signalling in such cells and for treating diseases associated with WIF1 under-expression.

Current tests make it possible to detect DNA mutations or the consequences of mutations (under-expression of a protein) or the presence of blood in the stools, with varying degrees of sensitivity and specificity.

The problem today is how to create a screening and/or prognosis test that will make it possible to discover and/or diagnose cancer in the early stages with high specificity and sensitivity, in a manner that is affordable in terms of its price.

This invention makes up for the drawbacks of the earlier art by offering a new method, process and kit for diagnosis/prognosis that make it possible to detect colorectal cancer for diagnosis, prognosis or therapeutic follow-up.

The process according to the invention particularly makes it possible to determine the possible presence of colorectal cancer, assess seriousness, identify the most suitable treatment and follow-up of treated patients.

This invention is aimed at remedying the drawbacks described above, and thus consists in a process or method for the diagnosis/prognosis and/or follow-up of colorectal cancer, including the following steps:
a. The extraction of nucleic material from a biological sample;
b. The use of at least one pair of amplification primers in order to obtain the amplicons of at least one target sequence;
c. The use of at least one detection probe to detect the presence of the said amplicons;
   characterised in that it includes a modification step between steps a) and b).

Advantageously, steps b) and c) are carried out at the same time and step b) is a conventional or quantitative PCR.

The detection probe according to the invention may include a marker. Preferably, the said target sequence is included in the WIF1 gene.

More particularly, the said primer pair includes at least one amplification primer including at least 15 nucleotide patterns of a nucleotide sequence selected from SEQ ID nos. 1 to 10 and/or the said detection probe includes at least 15 nucleotide patterns of a nucleotide sequence selected from SEQ ID nos. 1 to 10.

More precisely, this invention also relates to an amplification primer for the diagnosis/prognosis and/or follow-up of colorectal cancer including at least 15 nucleotide patterns of a nucleotide sequence selected from SEQ ID nos. 1 to 10.

Similarly, this invention also relates to a detection probe for the diagnosis/prognosis and/or follow-up of colorectal cancer including at least 15 nucleotide patterns of a nucleotide sequence selected from SEQ ID nos. 1 to 10.

Advantageously, this invention concerns the use of the said amplification primer and/or the said detection probe for the diagnosis/prognosis and follow-up of colorectal cancer.

Advantageously, this invention also concerns a kit for the diagnosis/prognosis and follow-up of colorectal cancer including the said primer and/or the said detection probe.

The invention will be better understood in the light of the description below, relating to illustrative examples of this invention that are not intended to be exhaustive in any way, by reference to the drawings attached, wherein:

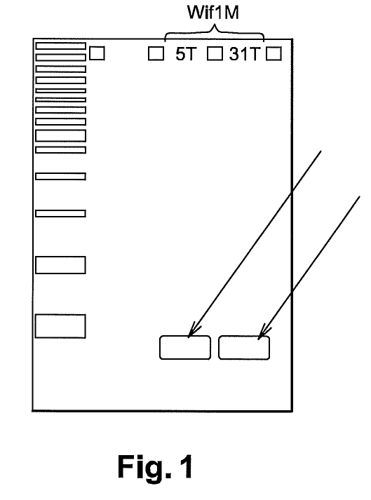

FIG. 1 is a representation of an agarose gel of sequences isolated from the tissue DNA of two patients (no. 5 cancer of the colon, no. 31 cancer of the rectum from a biological collection);

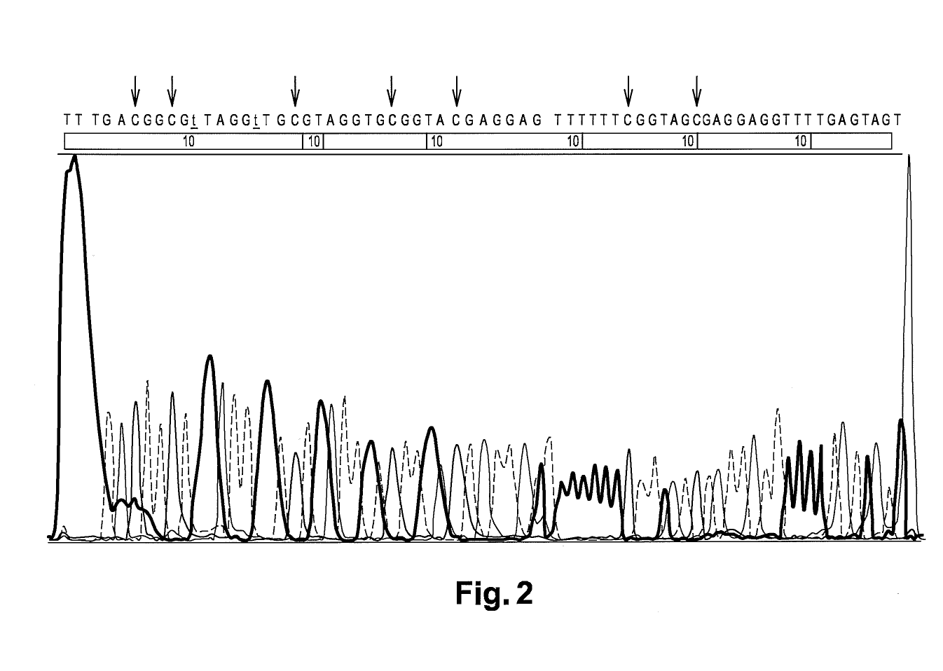

FIG. 2 is a chromatographic representation of the sequencing of the amplicon of gene WIF1 that also reveal CG methylation loci;

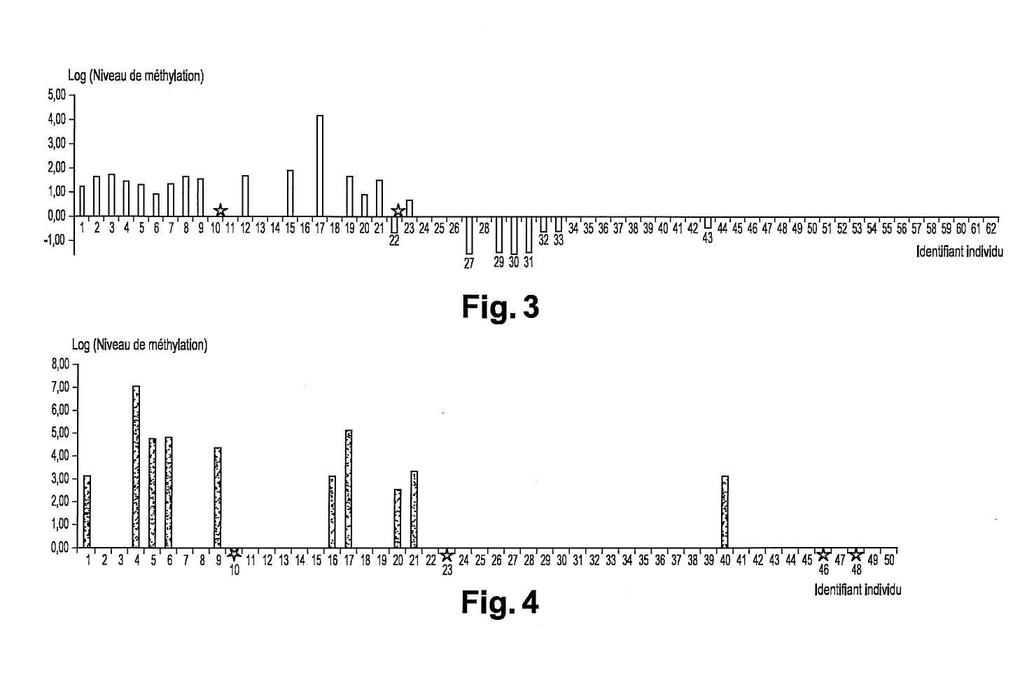

FIGS. 3 and 4 are graphical representations illustrating the methylation levels found in the urine and serum of individuals (normal or with colorectal tumours).

The inactivation of the APC gene leads to the activation of signalling pathways of proteins such as Wnt and the formation of early adenomas. It is known that the WIF-1 pathway is one of the genes involved in the inhibition of the Wnt signalling pathway.

This invention provides a simplified method, process, test and kit for diagnosis/prognosis that make it possible to detect colorectal cancer for diagnosis, prognosis or therapeutic follow-up based on the detection of functional DNA abnormalities such as methylations.

It is known that DNA alterations always originate in a tumour and that such DNA is released in the lumen of the colon, blood circulation or urinary vessels. The search for abnormalities, such as for example methylated DNA, makes it possible to detect a larger number of tumours, including colorectal tumours.

Also, it has been demonstrated that some phenotypes of epigenetic abnormalities with methylated CpG islands (regions where pairs of the C and G nucleotides are very frequent) are present in some colorectal cancers. Indeed, it has been described that the silent expression of WIF1 is due to a hypermethylated promoter.

On a preliminary basis, a “biological sample” is harvested from an individual likely to suffer from cancer or requiring screening/diagnosis/follow-up. That sample, which contains circulating cells and nucleic material, may be their blood, stools, saliva, urine, bile, serum, plasma etc.

The “nucleic material”, composed of sequences of deoxyribonucleic acid (DNA) or ribonucleic acid (RNA), collected from the said individual via the said biological sample includes a target sequence.

A “target sequence” is the specific nucleotide sequence of a target gene, whether single or double strand, such as WIF1.

For example, here is the sequence of the WIF1 gene:

aaagtgcagggattacaggcgtgagccatcgcgcccggccgaattcagc
aacttttaaaaaatatcagcaaacgtgaagatatccacgatgttagagg
agccctaccccggagggtcaggtacagcttcgtccagggcccagggcac
tcttccgggcactgccgggttatcagggagacagacgggaatccccaaa
tgctgggtgtcgggcaagtaccagctggacgccctgcgcctcgagccaa
ggccagcgctgccatcggcaccatcggacagtcgagcctcgagttttaa
ctgcttgggagcgcgcaaagtgccagcctatcgcagaacggagcgcata
gggttggcggagagaggaatcctactggctgaaagggagacgaaggcaa
tttgcgccttcagtgagcgccggaggaggaacaggagtcatcacctcat
catcatcatcatcatcatcatcaccatcaccatcaccatcatcagcact
cagtcaagcccagcgttgtctgcttccccatttccctcccccgaagcct
cccttggcccgaggaggtggcgagtgatgtcccaggggtctctgagtgc
ccttctccgggtccgccagccctacacgcccacttcgcgggcgctccac
tggggcaccgcactgtgaatgcagcctcgggggtccctcgcggccccgc
ccccgggggggccccacagcgcccccaagtggcggccgcccaggcctcg
cgggccccactcctcgctcgcacctcgctcgcgcagcccttcccgctct
tctgttctcgctctatttgccccgctgactgctggcctcgccagctttg
ccagtcttacgtctctgccgcccccactcccgcccgcgccccatcttct
tgcgcgactcgcgccgctggtccccccctcctcctcccgcgtcctgcct
gccccctcctcctgctctcgcaggctccttggcacccaggccgggaggc
gacgcgcccagccGTCTAAACGGGAACAGCCCTGGCTGAGGGGCTGCAG
CGCAGCAGAGTATCTGACGGCGCCAGGTTGCGTAGGTGCGGCACGAGGA
AGTTTTCCCGGCAGCGAGGAGGTCCTGAGCAGCATGGCCCGGAGGAGCG
CCTTCCCTGCCGCCGCGCTCTGGTCTGGAGCATCCTCCTGTGCCTGCTG
GCACTGCGGGCGGAGGCCGGGCCGCCGCAGGAGGAGAGCCTGTACCTAT
GGATCGATGCTCACCAGGCAAGAGTACTCATAGgtaaggcccccgcctc
caggccttcccctgcct

After it is modified by sodium bisulphite, the sequence of interest or target sequence of the fully methylated WIF1 gene is:

gtttagCGttgtttgttttttttatttttttttttCGaagttttttttg
gttCGaggaggtggCGagtgatgttttaggggtttttgagtgttttttt
tCGggttCGttagttttataCGtttatttCGCGggCGttttattgggCG
tatCGtattgtgaatgtagtttCGggggtttttCGCGgtttCGttttCG
ggggggttttatagCGtttttaagtggCGgtCGtttaggtttCGCGggt
tttatttttCGttCGtatttCGttCGCGttagtttttttCGtttttttg
ttttCGttttatttgtttCGttgattgttggtttCGttagttttgttag
ttttaCGtttttgtCGtttttattttCGttCGCGttttatttttttgCG
CGattCGCGttCGttggtttttttttttttttttCGCGttttgtttgtt
ttttttttttgttttCGtaggttttttggtatttaggtCGggaggCGaC
GCGtttagtCGTTTAAACGGGAATAGTTTTGGTTGAGGGAGTTGTAGCG
TAGTAGAGTATTTGACGGCGTTAGGTTGCGTAGGTGCGGTACGAGGAGT
TTTTTCGGTAGCGAGGAGGTTTTGAGTAGTATGGTTCGGAGGAGCGTTT
TTTTTGTCGTCGCGTTTTGGTTTTGGAGTATTTTTTTGTGTTTGTTGGT
ATTGCGGGCGGAGGTCGGGTCGTCGTAGGAGGAGAGTTTGTATTTATGG
ATCGATGTTTATTAGGTAAGAGTATTTATAGgtaaggttttCGttttta
gCGtttttttttgttt

A “nucleotide sequence” is a sequence of nucleotide patterns, that is to say a sequence of nucleic acids or polynucleotides or fragments of the same.

A “nucleotide pattern” is a natural nucleotide of nucleic acid or a modified nucleotide (base, phosphate, sugar).

The structures and modifications of the sequences are either natural or the result of a genetic recombination or chemical synthesis.

The process according to the invention relates to new nucleotide sequences of a target sequence, such as WIF1, which may be used as amplification primers or hybridisation probes in order to detect and/or provide a prognosis and/or follow up an individual suffering from colorectal cancer.

More particularly, the inventors have discovered that the said target nucleotide sequences have functional abnormalities such as methylations.

This invention thus provides a new method, process and kit for diagnosis/prognosis for detecting colorectal cancer in order to allow a diagnosis and/or prognosis and/or therapeutic follow-up by searching for the hypermethylation of a WIF1 gene or its promoter and by quantifying the abnormality.

More precisely, the said nucleotide sequence according to the invention including at least 15 nucleotide patterns of a sequence selected from SEQ ID nos. 1 to 10 is very relevant:
as an amplification primer to amplify the target sequences, such as the WIF1 gene,
as a hybridisation probe for specific hybridisation on the target sequences, such as the WIF1 gene.
   Step a) Consists in Extracting the Nucleic Material from a Biological Sample.

The detection of the presence of tumoral DNA from biological samples or effluent requires the extraction of total DNA. Such extraction is carried out by any protocol for nucleic acid extraction from biological samples of a type that is known in itself.

For example, such extraction may be carried out by subjecting the cells from the biological sample to lysis, using for instance a chemical, physical, thermal or other method.

Advantageously, this step may be followed by purification (centrifugation or any other method of purification of a type known in itself) consisting in separating and concentrating nucleic acids from the other constituents.

The cells that escape apoptosis, like tumour cells, have longer DNA fragments than those of normal cells. Long DNA may thus act as the first marker of colorectal tumours.

For example, the biological sample may be stool samples (5 to 10 g) placed in a buffer solution that favours the extraction of nucleic acids (DNA, RNA) using the Exact or Qiagen kit or any other method. The nucleic acids obtained may be suspended in an EDTA-enriched Tris buffer.

Of course, those skilled in the art know how to adapt the extraction, purification and conservation of nucleic acids to the biological samples from which they are derived.

### Step a b) is the Modification of the Extracted DNA.

In order to detect functional abnormalities in the target sequence, it is necessary to modify the DNA collected using any type of modification method of a type known in itself.

The methylation of a gene or its promoter influences its expression and may lead to its functional inactivation, for example.

The present inventors have searched the normality level from the main methylated markers, since the number of methylated genes and their level of methylation increase with age.

As an illustration, the collected DNA may be purified from T lymphocytes using the Qiagen kit and artificially methylated by methyltransferase or any other method. Then, the DNA methylated in this way is modified with sodium bisulphite before it is purified.

Sodium bisulphite transforms the non-methylated cytosine into uracil, while the methylated cytosine remains intact.

After this step, the methylated form of the WIF1 gene can be amplified and quantified.

### Steps b) and c) are: The Use of Amplification Primers so as to Generate Amplicons of at Least One Target Sequence of the Nucleic Material.

According to this invention, an “amplification primer” is a nucleic sequence including 10 to 100 nucleotide patterns, preferably 40 to 80 base pairs of at least one target sequence of genetic material.

Preferably, the said primer includes at least 15 nucleotide patterns of a sequence selected from:
a sequence from SEQ ID nos. 1 to 10;
a homologous sequence from SEQ ID nos. 1 to 10 that is complementary or sufficiently complementary or sufficiently homologous for hybridising with SEQ ID nos. 1 to 10 or their complementary sequences;
a sequence including a sequence from SEQ ID nos. 1 to 10, which has the same function as the amplification primer.

A sequence that is too small improves efficiency but turns out to be less specific, and conversely, a sequence that is too large reduces efficiency while it increases the specificity of the target sequence.

More precisely, the use of a pair of primers makes it possible to carry out a process of amplification through enzyme polymerisation (targeted in vitro replication technique called a Polymerase Chain Reaction or PCR), whereby large quantities of a specific DNA fragment of a definite length can be obtained from a sample.

These techniques are well known to those skilled in the art.

“Hybridisation” is a process whereby two nucleic sequences, such as for instance a primer and a target sequence, link in specific conditions that are well known to those skilled in the art.

The primer sequences used may be:

(SEQ ID no. 4)
WIF1 sense: TATACGTTTATTTCGCGGGC
(SEQ ID no. 5)
WIF1 antisense: ACGACGACCCGACCTCCGCCC
or:
(SEQ ID no. 6)
WIF1 sense CGTATTTCGTTCGCGTTAGTTTTTTTC
(SEQ ID no. 7)
WIF antisense CTCTCCTCCTACGACGACCCG

Alternatively, the primer sequences and/or probe sequences include at least 15 nucleotide patterns of a nucleotide sequence selected from:

(SEQ ID no. 1)
gtttagCGttgtttgttttttttatttttttttttCGaagttttttttg
gttCGaggaggtggCGagtgatgttttaggggtttttgagtgttttttt
tCGggttCGttagttttataCGtttatttCGCGggCGttttattgggCG
tatCGtattgtgaatgtagtttCGggggtttttCGCGgtttCGttttCG
tggggggttttatagCGtttttaagggCGgtCGtttaggtttCGCGggt
tttatttttCGttCGtatttCGttCGCGttagtttttttCGtttttttg
ttttCGttttatttgtttCGttgattgttggtttCGttagttttgttag
ttttaCGtttttgtCGtttttattttCGttCGCGttttatttttttgCG
CGattCGCGttCGttggtttttttttttttttttCGCGttttgtttgtt
ttttttttttgttttCGtaggttttttggtatt1taggtCGggaggCGa
CGCGtttagtCGTTTAAACGGGAATAGTTTTGGTTGAGGGAGTTGTAGC
GTAGTAGAGTATTTGACGGCGTTAGGTTGCGTAGGTGCGGTACGAGGAG
TTTTTTCGGTAGCGAGGAGGTTTTGAGTAGTATGGTTCGGAGGAGCGTT
TTTTTTGTCGTCGCGTTTTGGTTTTGGAGTATTTTTTTGTGTTTGTTGG
TATTGCGGGCGGAGGTCGGGTCGTCGTAGGAGGAGAGTTTGTATTTATG
GATCGATGTTTATTAGGTAAGAGTATTTATAGgtaaggttttCGttttt
agCGtttttttttgttt
(SEQ ID no. 2)
CGTTTAAACGGGAATAGTTTTGGTTGAGGGAGTTGTAGCGTAGTAGAGT
ATTTGACGGCGTTAGGTTGCGTAGGTGCGGTACGAGGAGTTTTTTCGGT
AGCGAGGAGGTTTTGAGTAGTATGGTTCGGAGGAGCGTTTTTTTTGTCG
TCGCGTTTTGGTTTTGGAGTATTTTTTTGTGTTTGTTGGTATTGCGGGC
GGAGGTCGGGTCGTCGTAGGAGGAGAGTTTGTATTTATGGATCGATGTT
TATTAGGTAAGAGTATTTATAG
(SEQ ID no. 3)
TTTGACGGCGTTAGGTTGCGTAGGTGCGGTACGAGGAGTTTTTTCGGTA
GCGAGGAGGTTTTGAGTAGT

Preferably, the sequences of the primers and probes used are:

(SEQ ID no. 8)
WIF1 sense: TTTGACGGCGTTAGGTTGC
(SEQ ID no. 9)
WIF1 antisense ACTACTCAAAACCTCCTCGCTACC
(SEQ ID no. 10)
WIF1 probe: CGGTACGAGGAGTTTT

For example, the methylation primers created are amplified by conventional or quantitative PCR on the lymphocyte matrix that is artificially methylated by the enzyme S-methyltransferase.

FIG. 1 is a representation of an agarose gel of the sequences isolated from tissue DNA of two patients (no. 5 cancer of the colon, no. 31 cancer of the rectum from a biological collection).

As illustrated in FIG. 1, the amplicons can then be analysed on agarose gel using electrophoresis in order to check that their sizes are indeed as expected for WIF1 primers.

In that way, it is possible to view the amplicons from the PCR in the expected weight range, corresponding to the 69 base pairs for WIF1 as indicated in FIG. 1 for a patient suffering from a colon tumour (no. T5) and another patient suffering from a rectal tumour (no. T31).

During the step when the target nucleic acids are detected, the use of a specific detection probe may be provided.

Of course, that direct or indirect detection step may be carried out using any other detection mode of a type known in itself.

A “hybridisation probe” is a nucleic sequence of 10 to 100 nucleotide patterns with hybridisation specificity in specific and definite conditions so that it hybridizes with the target nucleic sequence.

Advantageously, the hybridisation probe is a detection probe. Indeed, the hybridisation probe may include a marker for detection. The said “marker” is a tracer capable of locating, marking or distinguishing what is recognisable because of its physical or chemical properties (radioactivity, fluorescence, mass, colour, luminescence and others via optical, electrical and other detection) in very small quantities.

The functional primers are analysed with a fluorochrome or other fluorescent or “quencher” that links with the amplification product (double-strand DNA).

Thus, a hybridisation reaction can be detected between a detection probe and the target sequence.

Alternatively, the detection probe may be a molecular beacon detection probe.

The hybridisation probe may also be a capture probe, where the said probe is immobilised on a solid support by any appropriate means (of a type known in itself). The hybridisation reaction between the capture probe and the target sequence is then detected.

For detecting the hybridisation reaction, one may also use marked target sequences, directly or indirectly, of the target sequence.

Alternatively, the hybridisation step may be a step in which the target sequence is marked and/or cleaved.

In order to eliminate the primer dimers that are generated spontaneously, the sense and antisense primers may be linked with a specific TaqMan probe built using well known software.

In order to correct any possible enzyme efficiency variability, the inventors of this invention have standardised the expression of the target gene by determining a housekeeping gene, the expression of which is required and common in all individuals, through a ratio.

Alternatively, the amplification is carried out at the same time as the detection step.

Alternatively, the amplification is a quantitative PCR that is used to directly quantify the required target sequences.

FIG. 2 is a chromatographic representation of the sequencing of the amplicon of gene WIF1 that also shows the CG methylation loci (arrow at the top of the figure) of two tumours of two different patients after treatment with sodium bisulphite.

The arrows show the methylated cytosines recognised by the CpG dinucleotide sequences, covered by the WIF1 primers.

FIGS. 3 and 4 are graphical representations illustrating the methylation levels found in the urine and serum of normal individuals and individuals suffering from colorectal cancer.

The level of methylation is indicated by the height of the bars and the stars show abnormal methylation that is not quantifiable by this process or any other similar process.

The results explained below illustrate the comparison experiments carried out and are not limitative in any event.

TABLE 1
Comparison of samples taken from normal individuals and individuals
suffering from tumours of varying degrees of development*

Diagnosis

| | | | |
|---|---|---|---|
| Cancer | With adenome | Normal | Total |

Number of individuals

| | 109 | 42 | 121 | 272 |
|---|---|---|---|---|
| Tissues | 10 | 0 | 10 | 20 |
| Urine | 41 | 7 | 14 | 62 |
| Serum | 41 | 7 | 14 | 62 |
| Stools | 68 | 35 | 105 | 208 |
| Test total | 160 | 51 | 143 | 352 |

According to the coloscopy files and pathology files

TABLE 2
Characteristics of markers after the extraction
of DNA from the tissue or biological effluent

| DNA Marker | Tissues | Stools | Urine | Serum |
|---|---|---|---|---|
| Muted | | | | |
| Kras12tgt | ND | 208 | ND | ND |
| Kras12ggc | ND | 208 | | |
| Methylated | | | | |
| Alx4 | 10 | 208 | 62 | 62 |
| Vimentine | 10 | 208 | 62 | 62′ |
| \Ai IF1 | 10 | 208 | 62 | 62 |

TABLE 3
Comparison of serum and urine methylation of WIF1, ALX4 and vimentin
in the same individuals.

| | serum | | Urine | | Serum & Urine | |
|---|---|---|---|---|---|---|
| Genes | sensibility | specificity | sensibility | specificity | sensibility | specificity |
| Wifi 1 | 29.16% (14/48) | 100% (0/14) | 52.08% (25/48) | 92.85% (1/14) | 60.41% (29/48) | 92.85% (1/14) |
| ALX4 | 14.58% (7/48) | 100% (0/14) | 22.91% (11/48) | 100% (0/14) | 35.41% (17/48) | 100% (0/14) |
| Vim | 8.33% (4/48) | 100% (0/14) | 4.16% (2/48) | 100% (0/14) | 10.41% (5/48) | 100% (0/14) |

As illustrated in tables 1 to 3, the biological samples were taken from 272 individuals. The DNA was extracted from tissue (10 normal and 10 tumoral) and the urine, serum and stools were subjected to DNA extraction, modification and methylation PCR to identify the target gene WIF1, ALx4 and vimentin. Two Kras mutations were detected in the stools as a faecal control of dosage.

The WIF1 methylation marker was the most sensitive in urine and serum, with sensitivity of 52.08% and 29.16% respectively.

The combination of the two media (urine and serum) improved the test sensitivity of all the screening markers: WIF1 60.41%, ALX4 35.41% and vimentin 10.41% with no loss of specificity.

The sensitivity and specificity of the combined tests (Kras mutations and methylation) was 23% and 97% respectively in stools.

The values varied as follows as regards the sensitivity and specificity of the genes evaluated: Kras1 [9.61% (10/104), 97.16% (3/106)], Kras2 [6.73% (7/104) and 99.05% (1/106)], [Wif1 9.61% (10/104), 99.05% (1/106)], [ALX4 8.65% (9/104) and 99.05% (1/106)] and [Vim 0.96% (1/104); 100% 17 (0/106)]. As the only test of methylation, the specificity of WIF1 was 97% for the detection of colorectal tumours.

Even though it was combined with other molecular markers, its sensitivity was improved. A systematic analysis of WIF1 methylation as the only marker in serum and/or urine (or even stools) of the same person could offer a non-invasive method of colorectal cancer screening.

The results obtained by comparison with other markers demonstrate that the sensitivity and specificity of this method are considerably greater. Indeed, the overall sensitivity of the WIF1 methylation test was evaluated to be 58% and its specificity 100%.

Of course, the method and the test according to the invention can be combined with or include other molecular markers in order to further increase its sensitivity.

This invention makes it possible to improve the screening strategy and the treatment of early forms of cancer or precancerous forms.

1. A process or method for the diagnosis/prognosis and follow-up of colorectal cancer, including the following stages:
a. The extraction of nucleic material from a biological sample;
b. The modification of the extracted nucleic material;
c. The use of at least one pair of amplification primers in order to obtain amplicons of at least one target sequence; and
d. The use of at least one detection probe to detect the presence of the said amplicons;
wherein the biological sample contains circulating cells and nucleic material from at least one biological effluent, particularly selected from the group formed by stools, saliva, urine, bile, serum and/or plasma and in that the said target sequence is included in the WIF-1 gene.

2. A process or method according to claim 1, wherein the steps c) and d) are carried out at the same time.

3. A process or method according to claim 1, wherein the said step c) is a conventional or quantitative PCR.

4. A process or method according to claim 1, wherein the said detection probe includes a marker.

5. A process or method according to claim 1, wherein the said primer pair includes at least one amplification primer including at least 15 nucleotide patterns of a nucleotide sequence selected from SEQ ID nos. 1 to 10 and/or the said detection probe includes at least 15 nucleotide patterns of a nucleotide sequence selected from SEQ ID nos. 1 to 10.

6. An amplification primer for the diagnosis/prognosis and follow-up of colorectal cancer, for the amplification of a target sequence included in the WIF1 gene, including at least 15 nucleotide patterns of a nucleotide sequence selected from SEQ ID nos. 1 to 10.

7. A detection probe for the diagnosis/prognosis and follow-up of colorectal cancer, designed for detecting the methylations of a target sequence included in the gene WIF1, including at least 15 nucleotide patterns of a nucleotide sequence selected from SEQ ID nos. 1 to 10.

8. An amplification primer according to claim 6, wherein the amplification primer includes at least 15 nucleotide patterns of the nucleotide sequence SEQ ID no. 3 or at least 15 nucleotide patterns of one of the nucleotide sequences SEQ ID no. 8, SEQ ID no. 9 or SEQ ID no. 10.

9. The use of at least one amplification primer according to claim 6 for the diagnosis/prognosis and follow-up of colorectal cancer from different samples of biological effluent taken from at least one individual.

10. A kit for the diagnosis/prognosis and follow-up of colorectal cancer including at least one amplification primer according to claim 6.

11. A process or method according to claim 2 wherein the said step c) is a conventional or quantitative PCR.

12. A process or method according claim 2, wherein the said detection probe includes a marker.

13. A process or method according claim 3, wherein the said detection probe includes a marker.

14. A process or method according claim 11, wherein the said detection probe includes a marker.

15. A process or method according to claim 2, wherein the said primer pair includes at least one amplification primer including at least 15 nucleotide patterns of a nucleotide sequence selected from SEQ ID nos. 1 to 10 and/or the said detection probe includes at least 15 nucleotide patterns of a nucleotide sequence selected from SEQ ID nos. 1 to 10.

16. A process or method according to claim 3, wherein the said primer pair includes at least one amplification primer including at least 15 nucleotide patterns of a nucleotide sequence selected from SEQ ID nos. 1 to 10 and/or the said detection probe includes at least 15 nucleotide patterns of a nucleotide sequence selected from SEQ ID nos. 1 to 10.

17. A process or method according to claim 4, wherein the said primer pair includes at least one amplification primer including at least 15 nucleotide patterns of a nucleotide sequence selected from SEQ ID nos. 1 to 10 and/or the said detection probe includes at least 15 nucleotide patterns of a nucleotide sequence selected from SEQ ID nos. 1 to 10.

18. A detection probe according to claim 7, wherein the detection probe includes at least 15 nucleotide patterns of the nucleotide sequence SEQ ID no. 3 or at least 15 nucleotide patterns of one of the nucleotide sequences SEQ ID no. 8, SEQ ID no. 9 or SEQ ID no. 10.

19. The use of at least one detection probe according to claim 7 for the diagnosis/prognosis and follow-up of colorectal cancer from different samples of biological effluent taken from at least one individual.

20. A kit for the diagnosis/prognosis and follow-up of colorectal cancer including at least one detection probe according to claim 7.
